(19) United States
(12) Patent Application Publication
(10) Pub. No.: US 2026/0325597 A1
(43) Pub. Date: Oct. 22, 2026

(54) NUTRITIONAL COMPOSITION

(71) Applicant: CARGILL, INCORPORATED, Wayzata, MN (US)

(72) Inventor: Falk BRUESE, Drensteinfurt (DE)

(21) Appl. No.: 18/880,999

(22) PCT Filed: Jun. 28, 2023
(86) PCT No.: PCT/US2023/069231

§ 371 Date: Jan. 3, 2025

(30) Foreign Application Priority Data
Jul. 6, 2022 (EP) 22183441.9

(51) Int. Cl. A61K 36/28; A23L 33/00; A61K 31/20; A61K 31/201; A61K 31/202

(52) U.S. Cl. CPC A61K 36/28; A23L 33/40; A61K 31/20; A61K 31/201; A61K 31/202

(57) ABSTRACT

The present invention relates to a nutritional composition comprising high-palmitic sunflower oil and at least one other nutritional ingredient, wherein the high-palmitic sunflower oil is a sunflower oil having a fatty acid moiety comprising palmitic acid (C16) in an amount of from 20.0 to 45.0 wt. %, and wherein the total oil and/or fat content of the nutritional composition has a fatty acid moiety that is further comprising 1.0 to 8.0 wt. % of stearic acid (C18); 25.0 to 50.0 wt. % of oleic acid (C18.1); and 10.0 to 28.0 wt. %. of linoleic acid (C18.2). The present invention further relates to the use of the high-palmitic sunflower oil in a nutritional composition, and wherein the high palmitic sunflower oil is a mixture of a high-palmitic high-oleic sunflower oil and a high-palmitic high-linoleic sunflower oil.

### CROSS-REFERENCE TO RELATED APPLICATIONS

This application claims the benefit of European Application No. 22183441.9, filed Jul. 6, 2022, U.S. which is incorporated by reference herein in its entirety.

### FIELD OF THE INVENTION

The present invention relates to a nutritional composition comprising high-palmitic sunflower oil and at least one other nutritional ingredient.

### BACKGROUND OF THE INVENTION

Nutritional compositions are compositions that are developed to cover the nutritional needs, of specific groups of people, such as preterm infants, infants, toddlers, elderly people, athletes, or human subjects having nutritional deficiencies and/or having a deficient immune system.

Nutritional compositions for early life nutrition aim to resemble human milk as much as possible, since human milk is generally seen as the ideal source of nutrition during early life. Early life nutrition and more specifically infant formula are nutritional compositions for formula-fed infants allowing a comparable growth and development as an exclusively breastfed infant. For this reason, in the early life compositions, not only the main nutrients (proteins, lipids and carbohydrates), but also the trace elements (minerals, vitamins and etc.), must be carefully prepared to meet the infants' nutritional needs.

One aspect wherein the nutritional compositions tend to resemble human milk fat as close as possible is its fatty acid profile. For that, a combination of vegetable and animal oils and/or fats may be used, including bovine milk fat, palm oil, palm oil fractions or combinations of two or more thereof.

There is however a growing consumer trend for reducing or completely banning from their diet ingredients from animal origin. Consumers switch towards plant-based alternatives because of various reasons such as health concerns, environmental concerns, sustainability or animal welfare. Likewise, consumers also want to ban palm oil from their diet.

There is thus a need in the industry for nutritional compositions comprising alternative oils and/or fats such that the targeted fatty acid composition of the nutritional composition can be achieved. The present invention provides such a product.

### SUMMARY OF THE INVENTION

The present invention relates to a nutritional composition comprising high-palmitic sunflower oil and at least one other nutritional ingredient, wherein the high-palmitic sunflower oil is a sunflower oil having a fatty acid moiety comprising palmitic acid (C16) in an amount of from 20.0 to 45.0 wt. %, wherein the total oil and/or fat content of the nutritional composition has a fatty acid moiety that is further comprising:
1.0 to 8.0 wt. % of stearic acid (C18);
25.0 to 50.0 wt. % of oleic acid (C18.1); and
10.0 to 28.0 wt. %. of linoleic acid (C18.2).

The present invention further relates to the use of high-palmitic sunflower oil in a nutritional composition, wherein the high-palmitic sunflower oil is a sunflower oil having a fatty acid moiety comprising palmitic acid (C16) in an amount of from 20.0 to 45.0 wt. %, and wherein the high palmitic sunflower oil is a mixture of a high-palmitic high-oleic sunflower oil having a fatty acid moiety comprising oleic acid (C18.1) in a range of from 45.0 to 65.0 wt. %, and a high-palmitic high-linoleic sunflower oil having a fatty acid moiety comprising linoleic acid (C18.2) in a range of from 35.0 to 55.0 wt. % and the high palmitic sunflower oil is present in an amount of from 25 to 95 wt. % of the total oil and/or fat content of the nutritional composition

### DETAILED DESCRIPTION

The present invention relates to a nutritional composition comprising high-palmitic sunflower oil and at least one other nutritional ingredient. The term “high-palmitic sunflower oil” in the present invention is encompassing sunflower oils having a fatty acid moiety comprising palmitic acid (C16) in an amount of from 20.0 to 45.0 wt. %, from 22.0 to 40.0 wt. %, or from 25 to 34.7 wt. %, and wherein the total oil and/or fat content of the nutritional composition has a fatty acid moiety that is further comprising:
1.0 to 8.0 wt. % of stearic acid (C18);
25.0 to 50.0 wt. % of oleic acid (C18.1); and
10.0 to 28.0 wt. %. of linoleic acid (C18.2).

The term “nutritional composition” according to the present invention encompasses compositions that are developed to cover nutritional needs of a human subject. The human subject can be a preterm infant, an infant, a toddler, a juvenile, an adolescent, an adult or an elderly subject. Furthermore, human subjects targeted for the nutritional composition according to the invention may relate to specific groups of people, such as, but not limited to athletes, or human subjects having nutritional deficiencies and/or having a deficient immune system. The nutritional compositions may also be designed for human subjects suffering a more specific disease state such as cancer, chronic obstructive pulmonary disease, and later-stage kidney disease or others. Amongst others, nutritional compositions may be helpful for people who struggle with a loss of appetite, have difficulty in chewing, have trouble preparing balanced meals, and/or are recovering from surgery or illness. In the event that the nutritional composition is meant for a complete nutrition, it can provide a healthy balance of protein, carbohydrate, and/or fat.

The nutritional compositions according to the invention may be in the form of a liquid, as a ready-to-drink nutritional composition, or used in feeding tubes. It can also be in the form of a formula base, i.e. a powder or a concentrated liquid, to be dissolved in water or in another fluid for the preparation of a ready-to-drink nutritional composition. The nutritional composition may also be in the form of a pudding or a jelly, or in the form of a cookie or a snack bar, or in any other form. Preferably the nutritional composition is a ready-to-drink nutritional composition, or a powder.

Preferably the nutritional composition according to the present invention is an early life nutritional composition, i.e. a composition that is particularly suitable for human subjects of 0 to 36 months of age.

The early life nutritional composition according to the present invention may be an infant formula (first age), for infants from birth to age of 6 months; a follow-on formula (second age), for infants from an age of 6 to 18 months, or a growing-up formula (third age) for infants from an age from 1 to 3 years.

In a more preferred aspect of the invention, the early life nutritional composition is a follow-on formula or growing-up formula.

The early life nutritional composition according to the invention may also be a preparation for special medical purposes such as, but not limited to, hypoallergenic formulae prepared from partially hydrolysed proteins, to prevent infants with a high risk of milk protein allergy having an allergic reaction or a soy-based formulae prepared with soy protein isolates, for infants with lactose intolerance and/or galactosemia or for infants who are allergic to cow's milk proteins. Further examples of early life nutritional composition according to the invention prepared for special medical purposes are nutrient-dense formulae enhanced in proteins, fats, minerals and vitamins for premature infants or low-birth-weight and elemental formulae, using free amino acids instead of proteins or peptides, for infants who are allergic to hydrolysed protein or soy, and the like.

The high-palmitic sunflower oil is a mixture of 2 or more high-palmitic sunflower oils. In case of a mixture of 2 or more high-palmitic sunflower oils it is understood that the fatty acid moiety of the mixture of the 2 or more high-palmitic sunflower oils has a content of palmitic acid (C16) in a range of from 20.0 to 45.0 wt. %, from 22.0 to 40.0 wt. %, or from 25 to 34.7 wt. %.

The high-palmitic sunflower oil may be sourced from oilseeds of any sunflower (Helianthus annuus) line bearing oil which exhibits, following crushing and extraction, and in the absence of hydrogenation, or other chemical or physical modification, a fatty acid moiety having a suitable content of C16. Examples of suitable sunflower oilseed lines are, but are not limited to, sunflower oilseed line IG-1297M or QQ-3598-M (EP1185161), CAS-5 or CAS-12 (Serrano-Vega, M. (2005) Lipids, Vol. 40, p 369-374)

In one aspect of the invention the high palmitic sunflower oil is a mixture of a high-palmitic high-oleic sunflower oil and a high-palmitic high-linoleic sunflower oil

The term “high-palmitic high-oleic sunflower” oil is encompassing high-palmitic sunflower oil having a fatty acid moiety comprising oleic acid (C18.1) in a range of from 45.0 to 65.0 wt. %, from 48.0 to 62.0 wt. %, or from 50.0 to 60.0 wt. %. High-palmitic high-oleic sunflower oil may be sourced from oilseeds of a sunflower line such as, but not limited to, CAS-12, IG-1297M or QQ-3598-M or a mixture of two or more thereof. The term “high-palmitic high-linoleic sunflower oil” is encompassing high-palmitic sunflower having a fatty acid moiety comprising linoleic acid (C18.2) in a range of from 35.0 to 55.0 wt. %, from 37.0 to 52.0 wt. %, or from 40.0 to 50.0 wt. %. High-palmitic high-linoleic sunflower oil may be sourced from oilseeds of a sunflower line such as CAS-5 or the like.

The high-palmitic sunflower oil may be present in an amount of from 25 to 95 wt. %, from 30 to 90 wt. % or from 35 to 80 wt. % of the total oil and/or fat content of the nutritional composition.

Preferably the high-palmitic sunflower oil is a mixture of a high-palmitic high-oleic sunflower oil and a high-palmitic high-linoleic sunflower oil, whereby the ratio of oleic acid over linoleic acid (C18.1/C18.2) is in a range of from 0.5 to 8.0, from 1.0 to 7.5, or from 1.5 to 6.5.

In one aspect of the invention, the high-palmitic sunflower oil is present as free oil in the nutritional composition.

In another aspect of the invention, the high-palmitic sunflower oil is present in the form of isolated or enlarged sunflower oleosomes.

Oleosomes, also known as “oil bodies”, “lipid bodies”, “lipid droplets” or “spherosomes”, are pre-emulsified droplets or vesicles of oil stored in plant seeds and used as energy source for plant growth and metabolism. Oleosomes are typically extracted from cells by a process of grinding the seeds and subsequently washing, filtering and homogenizing the ground seeds to form an aqueous suspension. Said suspension is centrifuged to separate the oleosomes, called isolated oleosomes.

“Isolated oleosomes” as used in the present description means oleosomes that have been isolated/removed from their natural source of high-palmitic sunflower seeds. The term encompasses oleosomes isolated from a single oilseed line, as well as blends of oleosomes that are isolated from more than one oilseed line.

Typically, isolated high-palmitic sunflower oleosomes in the nutritional composition of the present invention may have an average globule diameter of about 1.5 to 2.5 micron.

“Enlarged oleosomes” as used in the present description means oleosomes that have been isolated/removed from their natural source and that have been subsequently subjected to a process for increasing their average globule size. Examples of processes for enlarging the average globule size of oleosomes are for example described in WO2021126408.

The enlarged high-palmitic sunflower oleosomes in the nutritional composition of the present invention may have an average globule diameter of the oleosomes in a range of from more than 2.5 to 15.0 micron, preferably from 3.0 to 12.0 micron, more preferably from 4.0 to 10.0 micron. Such a globule size is important when being used for an early life nutritional composition, since it mimics the average globule size of human breast milk of about 5.0 micron.

The average globule diameter of isolated or enlarged oleosomes is expressed as the D50-value (D50). The D50-value of oleosomes is the diameter below which 50% of the volume of oleosome particles lies, and it is expressed in micron (=micrometer, symbol: μm). To measure the average globule diameter (D50-value) of the oleosomes, the oleosomes are considered spherical and in case of non-spherical oleosomes, the diameter is considered as being the largest dimension that can be measured between two opposite points on the surface thereof.

To be able to measure the globule diameter of the oleosomes, a Mastersizer 3000 from Malvern equipped with a Hydro module may be used. A refractive index of 1.47 is used to measure the oleosomes size. The concentration of the oleosomes in the buffer is such that an obscuration in the range from 8.0 to 8.5% in the Mastersizer equipment is obtained. Obscuration within the Mastersizer is the amount of light blocked or scattered, by the particles. Therefore, the oleosomes are diluted in a buffer solution containing 10 mM sodium phosphate, pH 7.4, and 1.0 weight % sodium dodecyl sulphate (SDS). To give some guidance, about 0.2 weight % of oleosomes is diluted in the buffer solution and the dilution is further adjusted to obtain the aforementioned obscuration. Once this optimal obscuration is obtained, the globule diameter is measured, and the average globule diameter (D50) is calculated.

The nutritional composition may further comprise oils and/or fats other than the high-palmitic sunflower oil. Sources of such oils and/or fats other than the high-palmitic sunflower oil may be from vegetable and/or animal origin or a combination thereof. These sources do not comprise high-palmitic sunflower oil having a fatty acid moiety with a content of palmitic acid (C16) in a range of from 20 to 45 wt. %.

Sources of oils and/or fats other than the high-palmitic sunflower oil from animal origin include, but are not limited to milk fat, pork fat (lard), cattle and sheep fat (tallow), poultry fat, fish oil, krill oil and combinations of two or more thereof. Sources of these fats, other than the high-palmitic sunflower oil, from vegetable origin include, without any limitation, cocoa butter, corn oil, cottonseed oil, groundnut oil, linseed oil, olive oil, palm, palm olein, palm stearin, rapeseed oil, high-oleic canola oil, rice bran oil, safflower oil (also known as flaxseed oil), sesame oil, soybean oil, sunflower oil, including mid- and high-oleic varieties of sunflower, high-stearic sunflower oil, coconut oil, palm kernel oil, MCT compositions (i.e. Medium Chain Triglyceride with fatty acid chain lengths in range of C8 to C12), algae oil, yeast oil, and combinations of two or more thereof.

Oils, fats, or combinations of oils and/or fats other than the high-palmitic sunflower oil may be present to obtain the desired composition of fatty acids in the nutritional composition together with the high-palmitic sunflower oil. Especially a desired amount of mono- and poly-unsaturated fatty acids and/or a desired amount of mid- and short chain fatty acids is targeted.

In one aspect of the invention, the total oil and/or fat content of the nutritional composition has a fatty acid moiety comprising palmitic acid (C16) in an amount of from 15 to 30 wt. %, from 16 to 28 wt. %, or from 18 to 26 wt. %. Next to the high-palmitic acid sunflower oil, other sources of palmitic acid, that may be comprised in the nutritional composition are amongst others palm oil, cottonseed oil, cocoa butter, illipe fat; olein or stearin fractions of palm oil, cottonseed oil, cocoa butter or illipe fat; and blends of 2 or more thereof.

In a preferred aspect of the invention, the nutritional composition is substantially free from palm oil or components sourced from palm oil. Components sourced from palm oil are for example single or multiple fractionated oleins, single or multiple fractionated stearins, palm mid fractions, interesterified palm, interesterified palm fractions, hydrogenated palm, hydrogentated palm fractions, any combination of 2 or more thereof, and the like.

Next to the high-palmitic high-oleic acid sunflower oil, other vegetable sources of oleic acid C18.1, that may be comprised in the nutritional composition are amongst others high-oleic or mid-oleic sunflower oil, high-oleic canola oil, high-stearic high-oleic sunflower oil, olive oil, palm oil and olein fractions (single or multiple fractions) of palm oil, or a mixture of 2 or more thereof. Animal sources of oleic acid may be, but are not limited to, chicken fat or lard or a mixture of 2 or more thereof.

Next to the high-palmitic high-linoleic acid sunflower oil, other vegetable sources of linoleic acid C18.2, that may be comprised in the nutritional composition are amongst others sunflower oil, safflower oil, soybean oil, corn oil, canola oil, high-stearic high-linoleic sunflower oil, or a mixture of 2 or more thereof.

The total oil and/or fat content of the nutritional composition may have a fatty acid moiety that is further comprising caprylic (C8), capric (C10), lauric (C12) and myristic (C14) acid in a combined content (C8+C10+C12+C14) of up to 20 wt. %, from 10 to 18 wt. %, or from 12 to 16 wt. %.

Sources of caprylic (C8), capric (C10), lauric (C12) and/or myristic (C14) acid that may be comprised in the nutritional composition are typically lauric fats such as for example coconut oil, palmkernel oil; olein or stearin fractions of coconut oil or palmkernel oil; MCT compositions; cuphea oil, or a mixture of 2 or more thereof.

The total oil and/or fat content of the nutritional composition may have a fatty acid moiety that is further comprising alpha-linolenic acid (C18.3) in a range of from 0.5 to 4.0 wt. %, from 0.6 to 3.5 wt. %, or from 0.7 to 3.0 wt. %.

Sources of C18.3 that may be comprised in the nutritional composition are, but are not limited to flaxseed, canola oil, walnut oil or mixtures of 2 or more thereof.

The nutritional composition according to the invention comprises at least one nutritional ingredient other than the high-palmitic sunflower oil. Nutritional ingredients are ingredients that contribute to the caloric intake and/or provide micronutrients. Preferably, the at least one other nutritional ingredient of the nutritional composition according to the invention is selected from the group consisting of proteins, carbohydrates, minerals, trace elements, essential amino acids, essential fatty acids, vitamins, and mixtures of two or more thereof. More preferably, the at least one other nutritional ingredient is selected from the group consisting of proteins, carbohydrates, essential amino acids, essential fatty acids, vitamins, and mixtures of two or more thereof.

Sources of proteins may be from vegetable and/or animal origin. Protein sources from animal origin include, but are not limited to, lean meat, poultry, fish, eggs or dairy products like milk, yoghurt, cheese, whey proteins or caseinate. Protein sources from vegetable origin include, without any limitation, seeds, nuts, beans, legumes, such as lentils and chickpeas, grain and cereal-based products. Further examples of protein sources from vegetable origin are soy protein and pea protein. Sources of proteins may also be in form of protein isolates. Preferably, the at least one other nutritional ingredient of the nutritional composition according to the invention is a protein that is from vegetable origin.

Sources of carbohydrates may be mono-saccharides, disaccharides, oligosaccharides, and polysaccharides, their corresponding polyols and combinations of two or more thereof.

Monosaccharides include glucose, fructose, xylose or galactose. Non-limiting examples of di-saccharides, such as maltose, saccharose, lactose, isomaltulose and mixtures of two or more thereof.

Examples of suitable polyols may include, sorbitol, mannitol, xylitol, erythritol, lactitol, mixtures of two or more thereof and the like.

Oligosaccharides are saccharide polymers containing a small number, typically three to ten, monosaccharides. Non limiting examples of oligosaccharides are Fructo-oligosaccharides (FOS) and Galacto-oligosaccharides (GOS), xylo-oligosaccharides (XOS), arabino-xylo-oligosaccharides (AXOS), manno-oligo-saccharides (MOS), and mixtures of two or more thereof.

Polysaccharides comprise soluble and insoluble fibers. Polysaccharides can be glucose polymers such as starch and starch-derivatives, fructans derived from chicory or inulin, polydextrose, agar, galactomannans such as guar gum and locust bean gum, pectin, pectin derivatives, seaweed derived polysaccharides such as carrageenan, resistant starches, cereal fibres, fruit fibres and fibres of legumes, oat fibers and the like.

Vitamins are essential micronutrients that an organism needs in small quantities for the proper functioning of its metabolism. Essential nutrients cannot be synthesized in the organism, either at all or not in sufficient quantities, and therefore must be obtained through the diet. Vitamins required by human metabolism are vitamin A, including all-trans-retinol, all-trans-retinyl-esters, as well as all-trans-beta-carotene and other provitamin A carotenoids, vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid or folate), vitamin B12 (cobalamins), vitamin C (ascorbic acid), vitamin D (calciferols), vitamin E (tocopherols and tocotrienols), and vitamin K (quinones).

Minerals are chemical elements required as essential nutrients by organisms to perform functions necessary for life. The major minerals in the human body are calcium, phosphorus, potassium, sodium, and magnesium. The trace elements that have a specific biochemical function in the human body are sulfur, iron, chlorine, cobalt, copper, zinc, manganese, molybdenum, iodine, and selenium.

Essential amino acids are amino acids that cannot be synthesized de novo by the organism at a rate commensurate with its demand, and thus must be supplied in its diet. Examples of amino acids that cannot be synthesized by humans are phenylalanine, valine, threonine, tryptophan, methionine, leucine, isoleucine, lysine, and histidine.

The synthesis of other amino acids, such as arginine, cysteine, glycine, glutamine, proline, and tyrosine can be limited under special pathophysiological conditions, such as prematurity in the infant or individuals in severe catabolic distress.

Essential fatty acids are fatty acids that humans must ingest because the body requires them for good health but cannot synthesize them. Alpha-linolenic acid (an omega-3 fatty acid) and linoleic acid (an omega-6 fatty acid) are essential for humans. Further examples may include docosahexaenoic acid, arachidonic acid, eicosapentaenoic acid, and gamma-linolenic acid.

In one aspect of the invention the nutritional composition further comprises at least one non-nutritional ingredient.

Non-nutritional ingredients according to the invention are ingredients that do not substantially add to the caloric intake and/or do not substantially provide micronutrients. Examples of non-nutritional ingredients are flavors, colorants, emulsifiers, acid regulators such as citric acid or lactic acid, preservatives, and the like. The non-nutritional ingredients may be from a natural or synthetic origin.

In a preferred aspect of the invention, the nutritional composition does not contain ingredients from animal origin. In other words, the nutritional composition is a plant-based product.

The nutritional composition according to the present invention has a fatty acid moiety that is closely resembling the fatty acid profile of human milk fat. In order to mimic the level of palmitate of human milk fat in the nutritional composition, very limited sources of palmitate are suitable alternatives to palm oil and bovine milk fat. Available sources such as cocoa butter, illipe fat or cottonseed oil will result in a too high increase of saturated fatty acids, and/or are not allowed to be used in infant formula. It has been found that high-palmitic sunflower oil is a very good source of palmitate in the nutritional composition. Sunflower is an oilseed that is generally accepted for use in infant formula. Additionally, it will not result in a too high increase of saturated fatty acids. By using a mixture of high-palmitic high-oleic and high-palmitic high-linoleic sunflower oil, it is possible to closely match the palmitic acid target and also closely match the desired amount of oleic and linoleic acid in the nutritional composition.

Furthermore, the invention also related to the use of high-palmitic sunflower oil in a nutritional composition, wherein the fatty acid moiety of the high-palmitic sunflower oil is comprising palmitic acid (C16) in a range of from 20 to 45 wt. % and wherein the high palmitic sunflower oil is a mixture of a high-palmitic high-oleic sunflower oil having a fatty acid moiety comprising oleic acid (C18.1) in a range of from 45.0 to 65.0 wt. %, and a high-palmitic high-linoleic sunflower oil having a fatty acid moiety comprising linoleic acid (C18.2) in a range of from 35.0 to 55.0 wt. % and the high palmitic sunflower oil is present in an amount of from 25 to 95 wt. % of the total oil and/or fat content of the nutritional composition.

### Examples

### 1. Oil Blend

Oil blends of comparative samples 1 and 2, and of the examples according to the invention are prepared according to the compositions as stated in table 1.

Source of C16 in the oil blends is in the comparative examples 1 and 2 respectively palm oil and bovine milk fat. In the examples 1 and 2 according to the invention a blend of sunflower oil from 2 high-palmitic sunflower seed lines is used to replace palm oil or bovine milk fat as source of C16.

TABLE 1
Composition of oil blends

| | | | Examples | |
|---|---|---|---|---|
| | Comparative | | according to | |
| | examples | | the invention | |
| | 1 | 2 | 1 | 2 |
| Coconut oil | 17 | | 18 | 18 |
| Sunflower oil | 14 | 12 | 13 | |
| High-stearic high-linoleic sunflower | | | | 13 |
| oil CAS-3** | | | | |
| Rapeseed oil | 9 | 6 | 10 | 10 |
| High-oleic sunflower oil | 19 | 17 | | |
| Palm oil | 41 | | | |
| Bovine milk fat* | | 65 | | |
| High-palmitic sunflower oil CAS-12 | | | 52 | 52 |
| High-palmitic sunflower oil CAS-5* | | | 7 | 7 |

*Fatty acid composition of bovine milk fat according to Hageman, J. (2019) Internat. Dairy J., Vol. 92, 37-49.
**Fatty acid composition of high-stearic high-linoleic sunflower oil CAS-3, and high-palmitic sunflower oil CAS-5 and CAS-12 according to Serrano-Vega, M. (2005) Lipids, Vol. 40, p 369-374.

The fatty acid profile of the oil blends is shown in table 2. The composition of each oil blend is made such as to mimic human milk fat as closely as possible.

TABLE 2
Profile of the fatty acid moiety of the oil blends

| | Human | Comparative | | Example according | |
|---|---|---|---|---|---|
| | milk | examples | | to the invention | |
| | fat** | 1 | 2 | 1 | 2 |
| C8-C14 | 13.9 | 14.0 | 12.7 | 14.2 | 14.2 |
| C16 | 21.9 | 21.6 | 21.4 | 21.9 | 22.0 |
| C18 | 7.4 | 3.6 | 7.4 | 2.4 | 5.3 |
| C18.1 | 36.3 | 42.9 | 35.5 | 40.1 | 38.7 |
| C18.2 | 14.0 | 15.8 | 10.5 | 14.7 | 12.8 |
| C18.3 | 0.8 | 0.8 | 0.8 | 0.8 | 0.8 |
| SAFA | 43.2 | 39.2 | 41.6 | 38.5 | 41.7 |

**Fatty acid composition of human milk fat (Europe) according to Hageman, J. (2019) Internat. Dairy .J., Vol. 92, 37-49.

### 2. Nutritional Compositions

The oil blends as described in point 1 are subsequently used in the preparation of an infant formula according to the following recipe:
15% Soy protein concentrate
59% Maltodextrin
25% Oil blend according to point 1
0.5% Lecithin
QS Vitamins and minerals

### 3. Preparation of the Nutritional Compositions

The infant formulae are prepared according to standard “wet mix” process, known to the person skilled in the art.
The oil blend as described in point 1 is mixed with lecithin to obtain an oil phase. The oil phase is kept at a temperature of about 60° C.
A water phase is prepared by mixing the soy protein concentrate and the maltodextrin in water. The water phase is also kept at about 60° C.
An emulsion is prepared by mixing the water phase and the oil phase at a temperature of about 60° C., followed by a high-pressure homogenisation.
The emulsion is pasteurized. The heat-sensitive vitamin- and mineral mix is added after the heat treatment.
The emulsion is spray-dried and a powder is obtained.

1. A nutritional composition comprising high-palmitic sunflower oil and at least one other nutritional ingredient, wherein the high-palmitic sunflower oil is a sunflower oil having a fatty acid moiety comprising palmitic acid (C16) in an amount of from 20.0 to 45.0 wt. %, wherein the total oil and/or fat content of the nutritional composition has a fatty acid moiety that is further comprising:
1.0 to 8.0 wt. % of stearic acid (C18);
25.0 to 50.0 wt. % of oleic acid (C18.1); and
10.0 to 28.0 wt. %. of linoleic acid (C18.2).

2. The nutritional composition according to claim 1, and the high-palmitic sunflower oil is present in an amount of from 25 to 95 wt. % of the total oil and/or fat content of the nutritional composition.

3. The nutritional composition according to claim 1, wherein the total oil and/or fat content of the nutritional composition has a fatty acid moiety comprising 15 to 30 wt. % of palmitic acid (C16).

4. The nutritional composition according to claim 1, wherein the high palmitic sunflower oil is a mixture of a high-palmitic high-oleic sunflower oil having a fatty acid moiety comprising oleic acid (C18.1) in a range of from 45.0 to 65.0 wt. %, and a high-palmitic high-linoleic sunflower oil having a fatty acid moiety comprising linoleic acid (C18.2) in a range of from 35.0 to 55.0 wt. %.

5. The nutritional composition according to claim 1, wherein the ratio of oleic acid over linoleic acid (C18.1/C18.2) is in a range of from 0.5 to 8.0, from 1.0 to 7.5, or from 1.5 to 6.5.

6. The nutritional composition according to claim 1, wherein the total oil and/or fat content of the nutritional composition has a fatty acid moiety that is further comprising caprylic (C8), capric (C10), lauric (C12) and myristic (C14) acid in a combined content of up to 20 wt. %.

7. The nutritional composition according to claim 1, wherein the total oil and/or fat content of the nutritional composition has a fatty acid moiety that is further comprising 0.5 to 4.0 wt. % of alpha-linolenic acid (C18.3).

8. The nutritional composition according to claim 1, and the nutritional composition is substantially free from palm oil and/or components sourced from palm oil.

9. The nutritional composition according to claim 1, and the nutritional composition is a plant-based product.

10. The nutritional composition according to claim 1, wherein the at least one other nutritional ingredient is selected from the group of proteins, carbohydrates, essential amino acids, essential fatty acids, vitamins, or mixtures of two or more thereof.

11. The nutritional composition according to claim 1, wherein the high-palmitic sunflower oil is in the form of isolated and/or enlarged sunflower oleosomes.

12. The nutritional composition according to claim 11, wherein the high-palmitic sunflower oleosomes have an average globule diameter in a range of from more than 2.5 to 15.0 micron.

13. The nutritional composition according to claim 1 and the nutritional composition is an infant formula, a follow-on formula or a young child formula.

14. Use of high-palmitic sunflower oil in a nutritional composition, wherein the high-palmitic sunflower oil is a sunflower oil having a fatty acid moiety comprising palmitic acid (C16) in an amount of from 20 to 45 wt. % and wherein the high palmitic sunflower oil is a mixture of a high-palmitic high-oleic sunflower oil having a fatty acid moiety comprising oleic acid (C18.1) in a range of from 45.0 to 65.0 wt. %, and a high-palmitic high-linoleic sunflower oil having a fatty acid moiety comprising linoleic acid (C18.2) in a range of from 35.0 to 55.0 wt. % and the high palmitic sunflower oil is present in an amount of from 25 to 95 wt. % of the total oil and/or fat content of the nutritional composition.
